(19) United States
(12) Patent Application Publication
(10) Pub. No.: US 2006/0173249 A1
(43) Pub. Date: Aug. 3, 2006

(54) Method of producing a synthesized bipolar ECG waveform from a scalar ECG waveform

(75) Inventor: Arthur R. Weeks, Oviedo, FL (US)

Correspondence Address: QUARLES & BRADY LLP, 411 E. WISCONSIN AVENUE, SUITE 2040, MILWAUKEE, WI, 53202-4497, US

(73) Assignee: Invivo Corporation

(21) Appl. No.: 11/271,256

(22) Filed: Nov. 10, 2005

Related U.S. Application Data
(60) Provisional application No. 60/643,027, filed on Jan. 11, 2005.

(51) Int. Cl. A61B 5/00

(52) U.S. Cl. 600/301

(57) ABSTRACT

An adapter converts a scalar ECG signal into a pseudo-vector ECG signal suitable for synchronization purposes on certain MRI systems having vector ECG inputs. The pseudo ECG signal while lacking significant diagnostic information replicates sufficient ECG features for timing purposes. Different pseudo ECG signals may be synthesized for compatibility with different vector ECG signals.

### CROSS-REFERENCE TO RELATED APPLICATIONS

This application claims the benefit of U.S. provisional application No. 60/643,027 filed Jan. 11, 2005, and hereby incorporated by reference.

### STATEMENT REGARDING FEDERALLY SPONSORED RESEARCH OR DEVELOPMENT

--

### BRIEF SUMMARY OF THE INVENTION

The present invention relates to electrocardiographic (ECG) systems, and in particular to an ECG system suitable for use in synchronizing the acquisition of data for magnetic resonance imaging (MRI) with the cardiac cycle.

Magnetic resonance imaging allows images to be created of soft tissue from faint electrical signals (nuclear magnetic resonance signals) emitted by nuclei of the tissue. The resonance signals are generated when the nuclei are subjected to a radio frequency pulse in the presence of a polarizing magnetic field.

A patient undergoing an MRI scan may be received into a relative narrow bore or cavity in an MRI magnet providing reduced access to the patient. Accordingly, it may be desirable to remotely monitor the patient's vital data, for example, heartbeat, respiration, temperature and blood oxygen, during the MRI scan, and provide a read-out of this data outside the magnet bore for review by an attending healthcare professional. The ECG signal used to monitor the patient's heartbeat may also be useful in synchronizing the acquisition of data in the MRI scan with the cardiac cycle. Such synchronization can reduce image artifacts caused by motion of the patient during the time required to acquire the necessary MRI data.

Generally, the acquisition of ECG signals in the MRI environment is hampered by the presence of rapidly switched radio frequency electrical and magnetic gradient fields used during the MRI acquisition process. Such electromagnetic fields introduce electrical noise to the ECG signal that can obscure clinical data and complicate the use of ECG signals for synchronization of the acquisition of MRI data.

One method of addressing the problem of electrical interference with the ECG signal when the ECG signal is used for synchronization is taught in U.S. Pat. No. 5,987,348 which employs a vector ECG signal instead of a traditional scalar ECG signal to provide additional resistance against some types of electrical noise.

As is understood in the art, scalar ECG is normally presented as a series of electrical voltages plotted against time. A scalar ECG may be acquired with as many as twelve leads (and thus mathematically presents a vector), but a vector angle and magnitude is not computed. In a sophisticated scalar ECG acquisition, electrodes are placed on both arms as well as both legs (and used in combination to obtain six signals) and supplemented with six chest leads. Nevertheless, acceptable ECG signals for some applications can be obtained with a subset of these leads for example: two electrodes, one on each of the arms and one electrode on one of the legs.

In contrast, a vector ECG signal is normally presented as a single vector having an angle and magnitude whose tip moves in three dimensions corresponding to orthogonal x, y and z-axes positioned with respect to the patient. A projection of the trajectory of the vector tip in a single plane may sometimes be used. A typical lead system for vector ECG acquisition is the Frank lead system in which electrodes are placed at the head, foot, right side of the chest, left side of the chest, sternum and the back, and on the chest at 45 degrees to the angles of the electrodes placed on the sternum and left side of the chest.

The requirement of a vector ECG acquisition, that leads be placed on the chest and back of the patient, is a problem in the MRI environment where the patient is typically supported in a supine position on a patient table and may need to have electrodes attached immediately before scanning. Conventional diagnostic ECG equipment for patient monitoring may not provide vector signals requiring a duplicate set of electrodes for monitoring and for synchronization. Finally, for remote monitoring systems that must transmit data on a limited set of leads or channels, the additional data required to transmit vector ECG signals adds an unnecessary complication to the transmission process.

### SUMMARY OF THE INVENTION

The present invention provides an adapter that accepts a conventional scalar ECG signal and converts it to a pseudo-vector ECG signal suitable for synchronizing MRI machines that expect vector ECG signal inputs. Generally, the invention identifies a synchronizing feature of the ECG waveform, for example the QRS complex, and synthesizes a pseudo-vector signal having the same timing characteristics, albeit with limited diagnostic content.

Specifically, the present invention provides a scalar to vector ECG adapter having input terminals for receiving ECG leads from ECG electrodes attached to a patient for acquisition of scalar ECG data. Detector circuitry detects at least one period feature of the scalar ECG signal indicating a predetermined point in a cardiac cycle, and a vector synthesizer creates and outputs a pseudo-vector ECG signal synchronized to the periodic feature for receipt by the MRI machine.

It is thus one object of at least one embodiment of the invention to provide the convenience of scalar ECG monitoring while still providing the necessary timing signals for synchronized MRI acquisitions.

The ECG may be selected from the left arm, right arm, right leg and left leg.

It is thus another object of at least one embodiment of the invention to provide a system that provides for both diagnostic and synchronizing ECG measurements with a simple electrode arrangement suitable for use with a patient on an MRI table or the like.

The periodic feature detected by the detector circuitry may be a QRS complex.

It is an object of at least one embodiment of the invention to provide a timing signal based on a prominent and relatively robustly detectable ECG feature.

The vector synthesizer may produce a pseudo-vector providing x- and y-signals.

It is thus another object of at least one embodiment of the invention to provide a simplified pseudo-vector that is nevertheless sufficient for vector sensing MRI equipment.

The vector synthesizer may produce a pseudo-vector signal providing binary signals. That is, the pseudo-vector signal may include a first signal having a first state during an occurrence of the periodic feature and a second state at other times and a second signal having the second state during the occurrence of the periodic feature and the first state at other times so that the first signal is an inverse of the second signal.

Thus, it is another object of at least one embodiment of the invention to provide an extremely simple representation of a vector signal that may be readily synthesized and that is highly resistant to noise and offset.

The adapter may provide a wireless transmitter and receiver interposed between the input terminals and the MRI machine and in particular, the wireless transmitter may transmit the scalar data.

It is thus another object of at least one embodiment of the invention to greatly simplify the data transmitted from the patient so as to facilitate wireless transmission. It is a further object of at least one embodiment of the invention to allow the use of standard wireless ECG monitors for both diagnostic and synchronization purposes.

The wireless transmitter may use a communication technique selected from the group consisting of radio, light, and acoustic transmission techniques so as not to provide interference with the operation of the MRI machine.

It is thus another object of at least one embodiment of the invention to provide a system that is compatible with the electrical environment of MRI.

These particular objects and advantages may apply to only some embodiments falling within the claims and thus do not define the scope of the invention.

### BRIEF DESCRIPTION OF THE DRAWINGS

FIG. 1 is a block diagram of a prior art vector ECG system used to synchronize the acquisition of MRI signals in an MRI machine;

FIG. 2 is a figure similar to that of FIG. 1 showing use of the present invention to convert at least one scalar ECG signal to a pseudo-vector signal to be provided to the MRI machine for synchronization of the MRI acquisition instead of a vector ECG signal;

FIG. 3 is a plot of amplitude as a function of time for a scalar ECG signal and for binary x- and y-components of the pseudo-vector signal generated by the present invention;

FIG. 4 is a plot of the y-component as a function of the x-component of the pseudo-vector signal of FIG. 3 superimposed on a similar plot of a conventional vector ECG signal (dotted lines), and further showing a detection zone used by the MRI machine in triggering an acquisition; and

FIG. 5 is a figure similar to that of FIG. 2 showing the interposition of a wireless transmitter and receiver in between the patient and the MRI machine.

### DETAILED DESCRIPTION OF THE PREFERRED EMBODIMENT

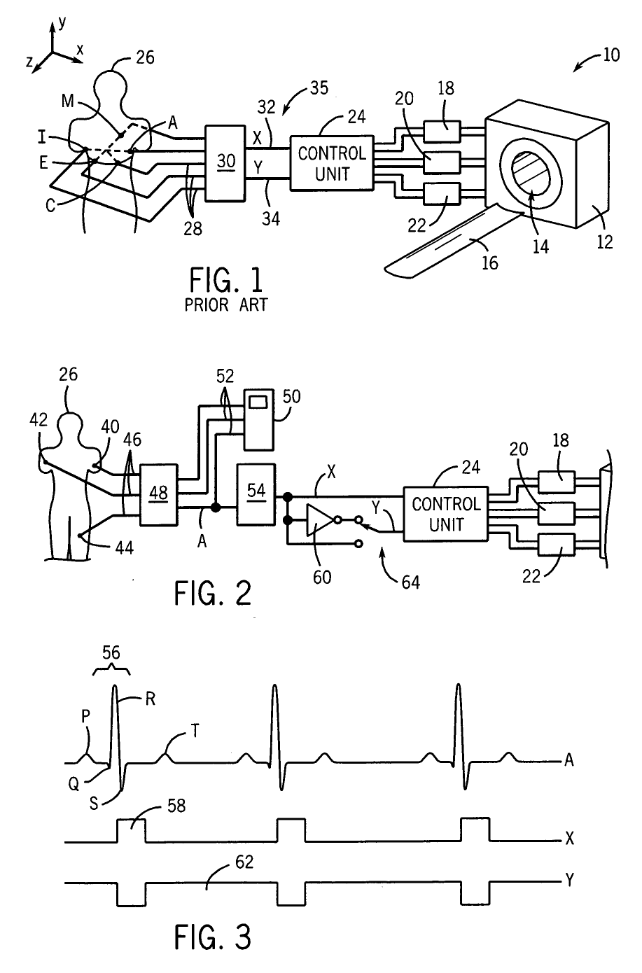

Referring now to FIG. 1, a conventional MRI machine 10 includes a magnet assembly 12 providing a polarizing magnet (typically 0.2 Tesla or higher) arranged around a bore 14 into which a patient 26 (for clarity, shown displaced to the left) may be supported on a patient table 16 moving into and out of the bore 14.

As is understood in the art, the magnet assembly 12 includes internal gradient magnet coils and a radio frequency (RF) antenna (not shown), the former driven by gradient system 18 and the latter driving by RF pulse system 20. The RF antenna also may be used to detect the NMR signal to provide a signal to NMR detection system 22. Each of these systems 18, 20 and 22 operate under the control of the control unit 24 executing a stored program to reconstructed MRI images as is understood in the art.

In the prior art, the patient 26 may have ECG electrodes I, E, C, A and M, per the well-known Frank configuration, arranged about the patient's chest for the acquisition of vector ECG data. Each of these electrodes connects via corresponding leads 28 to an amplifier/network 30 that produces a vector ECG signal 35 composed of an x-signal 32 and a y-signal 34 corresponding to the projection of the vector ECG along the x- and y-axes. Per convention, the x- and y-axes together define a frontal plane where the y-axis is vertical and the x-axis is horizontal.

The x-signal 32 and y-signal 34 are received by the control unit 24 which processes the vector ECG signals to detect a periodic feature that may be used to coordinate the acquisition of MRI images (by controlling the timing of the signals produced by systems 18 and 20) with a particular phase of the cardiac cycle. U.S. Pat. No. 5,987,348, hereby incorporated by reference, describes such a prior art system and its operation.

Referring now to FIG. 2 in the present invention, the patient 26 may have ECG electrodes 40, 42, and 44 placed for the acquisition of scalar ECG data. In this example, ECG electrodes 40, 42, and 44 are placed on the left arm, the right arm and the left leg, respectively, but many other configurations may also be used. The ECG electrodes are combined by network/amplifier 48 to form signals 52 on leads I, II and III according to the following table:

Lead I = left arm (electrode 40)-right arm (electrode 42)
Lead II = left leg (electrode 44)-right arm (electrode 42)
Lead III = left leg (electrode 44)-left arm (electrode 40)

Referring still to FIG. 2, the network/amplifier 48 provides amplification and filtering as necessary and as is known in the art, and transmits one or more of the signals 52 of the leads to a remote patient monitoring unit 50 that may be used to observe diagnostic ECG information reflecting patient health.

As will be described in further detail below, at least one of the signals 52 (designated A) is also transmitted to a detector 54 of the present invention which will synthesize a pseudo-vector signal 65 compatible with the synchronizing inputs of the control unit 24.

As shown in FIG. 3, this signal A may, for example, provide a positive going QRS complex 56 occurring at periodic intervals and identifying a particular phase of the cardiac cycle. Typically, the QRS output will be in the range of zero to 1 millivolt and 20 milliseconds in duration for the QRS complex. The size and distinctive nature of the QRS complex makes it particularly suitable for use as a synchronization signal and accordingly in a preferred embodiment, the detector 54 produces a first signal X being a binary signal having an upward pulse 58 aligned with the QRS complex 56.

Automatic identification and extracting of the timing of the QRS complex 56 is understood in the art and may, for example, be done with a thresholding circuit looking at a voltage threshold of the normalized signal A, or through more sophisticated correlation analysis where the waveform A is correlated on a rolling basis to a standard QRS complex, or adaptive filtering or other techniques known in the art.

This signal X may also be provided to an inverter 60 which produces signal Y, that is, the logical inverse of signal X, signal Y having an upward pulse 62 during periods of time when upward pulse 58 does not occur.

The X and Y signals produced by the detector 54 together form a pseudo-vector signal and may be provided to the control unit 24 in place of the x- and y-components of a true vector ECG.

Generally, a two-dimensional vector ECG waveform may contain components that have two positive QRS complexes (first mode) or one positive and one negative QRS complex (second mode) depending on how the ECG electrodes are placed on the human body. Accordingly, the present invention provides a switch 64 so that the inverter 60 may be bypassed so that the X and Y signals can both be of identical polarity. In this way, a pseudo-vector signal can be created to simulate vector ECG signals associated with different vector configurations required by MRI machines.

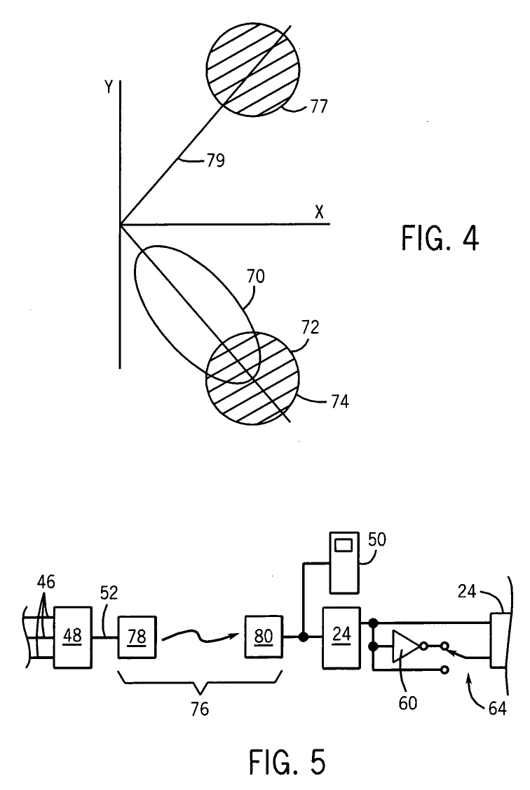

Referring now to FIG. 4, an example vector ECG signal of the second mode will provide a loop trajectory 70 that passes over the x-y plane into and out of a detection zone 72 used by the control unit 24 to detect the given periodic feature of the cardiac cycle. A first pseudo-vector signal of the present invention in which the X and Y signals are logical inverses produces a line trajectory 74 also passing into and out of the detection zone 72 to provide for the necessary triggering.

For alternative detection zone 77 for a vector ECG of a second mode, switch 64 may be used to bypass the inverter 60 to produce trajectory 79. In this example, the pseudo-vector ECG signal eliminates T, P and U waves, however, in an alternative embodiment, the T, P and U waves may be detected for creation of the pseudo ECG signal.

Referring now to FIG. 5, a single patient monitor may be placed on the patient 26 when the patient is in the bore 14 to communicate remotely with the control unit 24 and a patient monitoring unit 50 for diagnostic monitoring. The problems of obstructions, caused by cabling and of electrical interference induced in the cable runs, may be avoided through the use of a wireless transmitter system 76 comprising a transmitter 78 and receiver 80. The transmitter 78 receives one or more of the signals 52 to transmit them to the receiver 80 which provides them to the patient monitoring unit 50. One such system is described in U.S. patent application Ser. No. 11/075,620 filed Mar. 9, 2005 hereby incorporated by reference.

In the present invention, one of the scalar signals from this wireless system as transmitted by the transmitter 78 may be also provided to the detector 54 allowing such wireless monitors to be used not only for diagnostic imaging, but also for timing purposes without the burden of wirelessly transmitting vector ECG data.

More generally, the transmitter system 76 need not be limited to a radio system, but may be an optical transmission system using fiber or free space optical transmission, an acoustic transmission system, or other transmission systems known in the art for transmitting information without interference with the MRI acquisition. In this context, the transmission of scalar data can facilitate the transmission process by reducing the bandwidth required for the transmission and/or increasing potential noise rejection.

It is specifically intended that the present invention not be limited to the embodiments and illustrations contained herein, but include modified forms of those embodiments including portions of the embodiments and combinations of elements of different embodiments as come within the scope of the following claims.

We claim:

1. A scalar to vector ECG adapter comprising:
input terminals for receiving ECG leads from ECG electrodes attached to a patient for acquisition of scalar ECG data;
detector circuitry detecting at least one periodic feature of the scalar ECG signal indicating a predetermined point in a cardiac cycle; and
a vector synthesizer creating and outputting pseudo-vector ECG signals synchronized to the periodic feature for receipt by an MRI machine.

2. The adapter of claim 1 wherein the ECG leads are selected from the right arm, left arm, right leg and left leg leads.

3. The adapter of claim 1 wherein the periodic feature detected by the detector circuitry is a QRS complex.

4. The adapter of claim 1 wherein the vector synthesizer produces a pseudo-vector providing x and y signals.

5. The adapter of claim 1 wherein the vector synthesizer produces a pseudo-vector signal providing binary signals, a first signal having a first state during an occurrence of the periodic feature and a second state at other times, and a second signal having the second state during the occurrence of the periodic feature and the first state at other times, so that the first signal is an inverse of the second signal.

6. The adapter of claim 1 wherein further including a wireless transmitter and receiver interposed between the input terminals and the MRI machine.

7. The adapter of claim 6 wherein the wireless transmitter receives the scalar data and the wireless receiver transmits the scalar data to the detector.

8. The adapter of claim 6 wherein the wireless transmitter and receiver communicate without interference to an operation of the MRI machine using a communication technique selected from the group consisting of radio, light, and acoustic transmission techniques.

9. In an MRI machine providing synchronization of an MRI acquisition to a cardiac cycle of a patient being image per a vector ECG input provided to vector input terminals of the MRI machine, an adapter comprising:
input terminals for receiving scalar ECG data;
detector circuitry detecting at least one periodic feature of the scalar ECG signal indicating a predetermined point in a cardiac cycle; and
a vector synthesizer creating and outputting pseudo-vector ECG signals synchronized to the periodic feature to the vector input terminals.

10. A method of synchronizing an MRI acquisition to a cardiac cycle comprising the steps of:
a) attaching ECG leads to a patient to acquire patient signals;
b) deducing a scalar ECG signal from the patient signals;
c) detecting at least one periodic feature of the scalar ECG signal indicating a predetermined point in a cardiac cycle; and
d) synthesizing and outputting pseudo-vector ECG signals synchronized to the periodic feature for receipt by an MRI machine.

11. The method of claim 10 wherein the ECG leads are attached at points selected from a patient's left arm, left arm, right leg and left leg.

12. The method of claim 10 wherein the periodic feature detected is a QRS complex.

13. The method of claim 10 wherein only x and y signals are synthesized.

14. The method of claim 10 wherein a pseudo-vector ECG signal is binary signals, a first signal having a first state during an occurrence of the periodic feature and a second state at other times, and a second signal having the second state during the occurrence of the periodic feature and the first state at other times, so that the first signal is an inverse of the second signal.

15. The method of claim 10 further including the step of wirelessly transmitting at least one of the patient signals, the scalar ECG signal, and the pseudo-vector signal.

16. The method of claim 15 wherein the wireless transmission uses a communication technique selected from the group consisting of radio, light, and acoustic transmission techniques.
